(19) United States
(12) Patent Application Publication
(10) Pub. No.: US 2014/0358041 A1
(43) Pub. Date: Dec. 4, 2014

(54) ASSESSING PHYSICAL STABILITY OF A PATIENT USING AN ACCELEROMETER

(71) Applicants: Matthew Alan Hopcroft, San Francisco, CA (US); Jerome Rolia, Kanata (CA); Sharad Singhal, Belmont, CA (US); Charles Edgar Bess, Frisco, TX (US); Henri J. Suermondt, Sunnyale, CA (US)

(72) Inventors: Matthew Alan Hopcroft, San Francisco, CA (US); Jerome Rolia, Kanata (CA); Sharad Singhal, Belmont, CA (US); Charles Edgar Bess, Frisco, TX (US); Henri J. Suermondt, Sunnyale, CA (US)

(21) Appl. No.: 14/372,416

(22) PCT Filed: Apr. 18, 2012
(86) PCT No.: PCT/US12/34131

§ 371 Date: Jul. 15, 2014

(51) Int. Cl. A61B 5/11

(52) U.S. Cl. CPC A61B 5/1117; A61B 5/1126
USPC 600/595

(57) ABSTRACT

A system includes an accelerometer worn by a patient to capture physiological data of the patient and transmit the physiological data and a processing system to receive the physiological data and assess a physical stability of the patient using the physiological data.

### BACKGROUND

Healthcare providers are often concerned with the physical stability of their patients. If a patient lacks physical stability, the patient may be susceptible to falls or other injuries which could lead to a permanent decline in function for some patients. The diagnosis of patient stability generally involves teams of healthcare specialists who assess individual patients. These assessments may be expensive and typically occur under controlled conditions. The assessments may not reflect a patient's time varying physical stability based on factors such as medication usage and tiredness, for example, and they may not provide for continuous assessment in a patient's own environment. In addition, a patient may not always be reassessed when a patient's condition changes. As a result, patients may not be accurately diagnosed for being at risk of falls or other injuries based on a lack of physical stability.

### BRIEF DESCRIPTION OF THE DRAWINGS

FIGS. 1A-1B are diagrams illustrating embodiments of a system for assessing physical stability of a patient using an accelerometer.

FIG. 2 is a flow chart illustrating one embodiment of a method for assessing physical stability of a patient using an accelerometer.

FIG. 3 is a signal diagram illustrating one embodiment of physiological data captured by an accelerometer.

FIG. 4 is a flow chart illustrating one embodiment of a method for assessing physical stability of a patient using physiological data captured by an accelerometer.

FIG. 5 is a block diagram illustrating one embodiment of a system for assessing physical stability of a patient using multiple accelerometers and/or other sensors.

### DETAILED DESCRIPTION

In the following detailed description, reference is made to the accompanying drawings, which form a part hereof, and in which is shown by way of illustration specific embodiments in which the disclosed subject matter may be practiced. It is to be understood that other embodiments may be utilized and structural or logical changes may be made without departing from the scope of the present disclosure. The following detailed description, therefore, is not to be taken in a limiting sense, and the scope of the present disclosure is defined by the appended claims.

As described herein, a system and method for monitoring of patients to assess physical stability is provided. Each patient wears a highly sensitive accelerometer that captures physiological data of the patient and transmits the physiological data to a processing system where the physiological data represents internal and external body movements of patient. The processing system receives the physiological data and assesses the physical stability of the patient using the physiological data. The processing system notifies the patient and/or a person associated with the patient (e.g., a healthcare provider or family member) in response to determining that the physical stability indicates a risk of the patient falling.

The embodiments described herein allow information about a patient to be inferred in a healthcare facility, a home, or another location from the physiological data of the patient provided by the accelerometer worn by the patient. The highly accelerometer automatically and continuously collects physiological data about a patient to provide increased opportunities to preemptively assess and avert falls by the patient. The sensitivity of the accelerometer is such that specific features and characteristics of physiological conditions of the patient can be observed (e.g., the cardiac pulse of the patient). This physiological data may be analyzed using both time-domain and frequency-domain information. This physiological data may also be correlated to events of interest as observed by healthcare providers in controlled conditions and/or patients in un-controlled conditions such as a patient's home.

As used herein, physiological data refers to a set of data values that collectively represent the frequency and amplitude of the movements and vibrations generated by the internal and external body functions of a patient over time and captured by a highly sensitive accelerometer. The physiological data represents internal and external body movements of patient. The human body can be described as a mechanical system with thousands of moving parts where every one of the parts can create a mechanical vibration from muscle movements or other body functions. These mechanical vibrations result from internal body functions such as the rise and fall of a chest during breathing and the beating of a heart. These mechanical vibrations also result from external motions such as muscle tremors or turning over during sleep. Many of the vibrations of a body are extremely small and/or occur slowly. From the physiological data, a processing system may determine a range of health information including heart rate, respiration rate, and other specific features in the physiological data in addition to the physical stability assessment described herein.

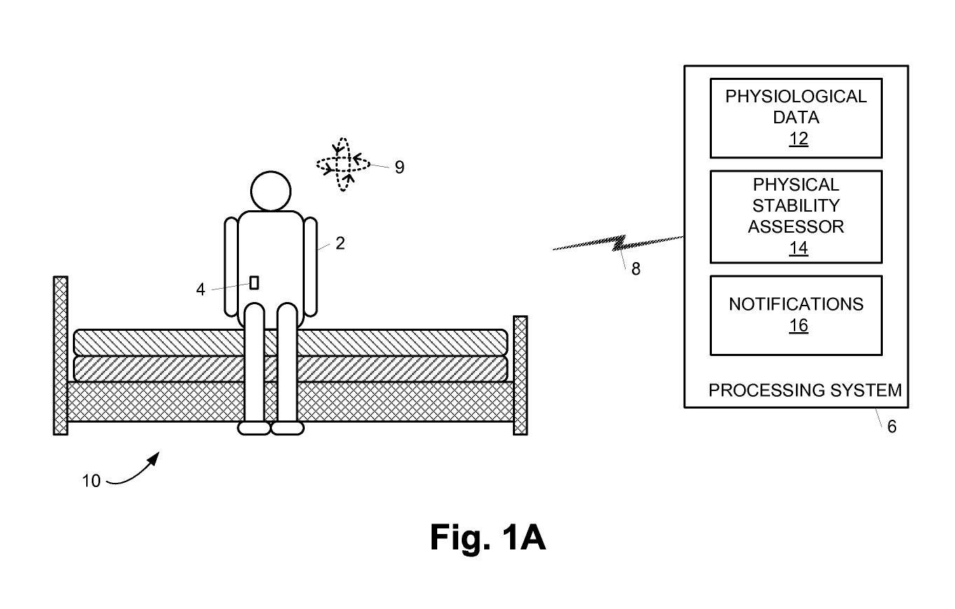

FIG. 1A is a schematic diagram illustrating an embodiment of a system 10 for assessing physical stability 9 of a patient 2 using an accelerometer 4 worn by patient 2. System 10 includes a processing system 6 that receives physiological data 12 from accelerometer 4 and processes physiological data 12 using a physical stability assessor 14 to generate notifications 16.

Patient 2 may be in any suitable location including healthcare facility, a home, or another location and may wear accelerometer 4 in any suitable way that allows accelerometer 4 to capture physiological data 12 of patient 2. Because of the high sensitivity of accelerometer 4, as described in additional detail below, the physiological data captured by accelerometer 4 typically transmits through any materials in direct or indirect contact with the body of patient 2 such as clothing and any support apparatus (not shown) for accelerometer 4. Additional details regarding determining an optimal placement of accelerometer 4 on patient are described below.

Accelerometer 4 captures physiological data 12 of patient 2 and provides physiological data 12 to a processing system 6 using any suitable wired or wireless connection 8 for transferring data between accelerometer 4 and processing system 6. Accelerometer 4 includes ultra-high sensitivity microfabricated accelerometer technology with three-phase sensing as described by U.S. Pat. Nos. 6,882,019, 7,142,500, and 7,484,411 and incorporated by reference herein in their entirety. Accelerometer 4 is a sensor which detects acceleration, i.e., a change in a rate of motion, with a high sensitivity and dynamic range. Because of the three-phase sensing technology, accelerometer 4 may sense acceleration levels as low as 10's of nano-gravities (ng) and may be manufactured and housed in a device that has typical dimensions of 5×5×0.5 mm or less using Micro-Electro-Mechanical-Systems (MEMS) technology. The combination of high sensitivity and small device size enabled by three-phase sensing techniques allows accelerometer 4 to capture physiological data 12 from patient 2. In addition, the sensitivity of accelerometer 4 allows processing system 6 to discern specific features of physiological conditions of patient 2 from physiological data 12. These features include not only cardiac pulse and respiratory rate but specific conditions such as arrhythmia. Additional details of accelerometer 4 are shown and described with reference to FIG. 1B below.

Processing system 6 periodically or continuously receives physiological data 12 from accelerometer 4 and processes physiological data 12 with a physical stability assessor 14 to assess physical stability 9 of the patient using physiological data 12. Physical stability 9 of the patient refers to the ability of patient 2 to maintain control of his or her body while moving. For example, patient 2 may reveal a lack of control of his or her body by stumbling, slipping, or having some other irregular motion when walking across a room. Processing system 6 examines physiological data 12 to determine whether physiological data 12 provides evidence of stable and/or unstable motion of patient 2. If processing system 6 determining that the physical stability of patient 2 indicates a potential for patient 2 to fall, processing system 6 generates a notification 16 corresponding to the physical stability that may be provided directly to patient 2 and/or to a person associated with patient 2 such as a healthcare provider or family member. In particular, processing system 6 may notify patient 2 that patient 2 may be at risk of falling and advise patient 2 to rest or seek care. Processing system 6 may further ask patient 2 for confirmation that patient 2 is ok and notify a healthcare provider if the confirmation is not received. Processing system 6 may discern a general trend towards more frequent irregular motions of patient 2 that may warrant further assessment by healthcare providers. In addition, processing system 6 may identify patterns of irregular motions based on the time of day or other information to help determine whether changes in behavior can be made by patient 2 to further avoid the risk of falls.

In one example, processing system 6 may determine patient 2 to be at risk of having their blood pressure drop suddenly if they get up after taking certain medications. The rapid drop in blood pressure may cause patient 2 to become dizzy and fall. Processing system 6 may infer a difference in patient motion between dizzy and non-dizzy scenarios while patient 2 is getting up and warn patient 2 not to get up if a dizzy scenario is detected.

Connection 8 includes any suitable type and combination of wired and/or wireless connections that allow accelerometer 4 to provide physiological data 12 to processing system 6.

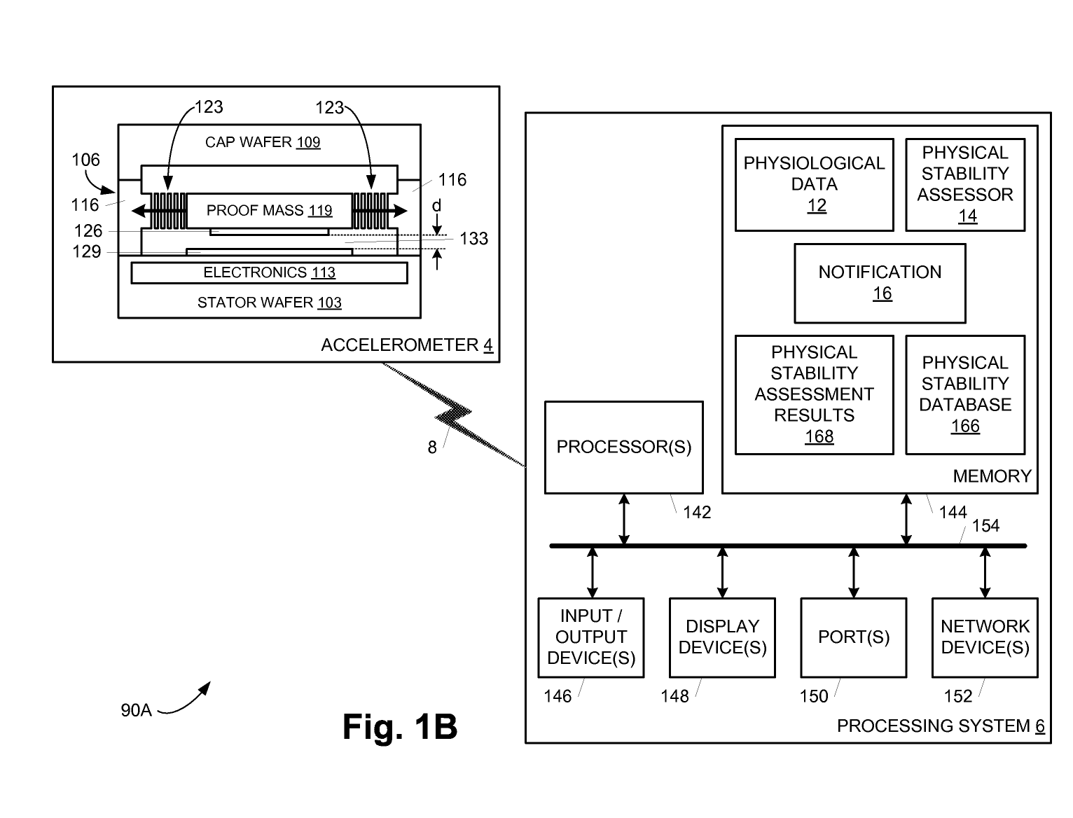

The functions of system 10 are further illustrated in FIG. 2 which is a flow chart illustrating one embodiment of a method for assessing physical stability of patient 2 using accelerometer 4.

In FIG. 2, accelerometer 4 captures physiological data 12 of patient 2 with an accelerometer 4 worn by patient 2 as indicated in a block 42. Accelerometer 4 may provide physiological data to processing system 6 by continuously transferring the data to the processing system or by storing the data in computer readable medium (not shown) for periodic transmittal or retrieval by processing system 6.

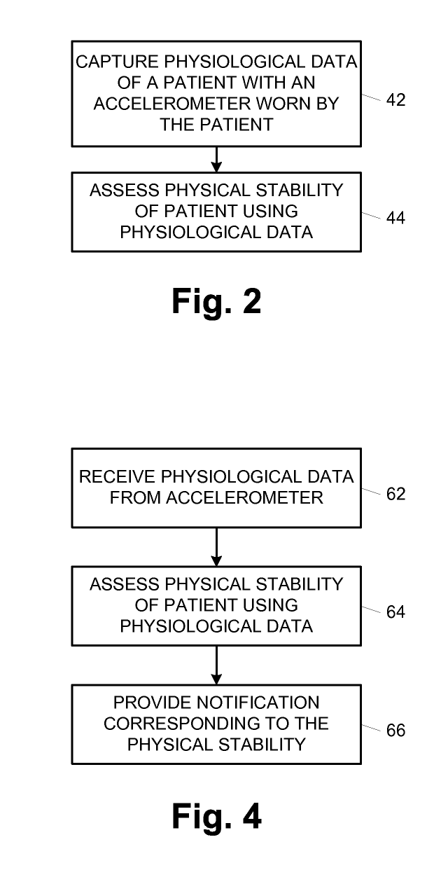

FIG. 3 is a signal diagram illustrating one embodiment of physiological data 12 captured by accelerometer 4. The amplitude of the vibrations of physiological data 12 is plotted in the y axis over time in the x axis. Various features of the health condition and physical stability of patient 2 appear in physiological data 12. For example, the pulse rate is apparent in a portion 12A of data 12 and actions of patient 2 such as rolling over, exiting a bed, and folding blankets on the bed are apparent in portions 12B, 12C, and 12D of data 12, respectively. Other information about patient 2 as well as any events and actions occurring around patient 2 may also be evident in physiological data 12. The typical level of the vibrations detected by accelerometer 4 in this example is a few μg (i.e., 1×10⁻⁶ g). Accordingly, physiological data 12 includes features present only in data captured by a high sensitivity accelerometer 4.

Referring back to FIG. 2, processing system 6 assesses the physical stability of patient 2 using physiological data 12 as indicated in a block 44. Processing system 6 analyzes physiological data 12 using time and/or frequency domain information and may correlate physiological data 12 to specific health conditions of patient 2.

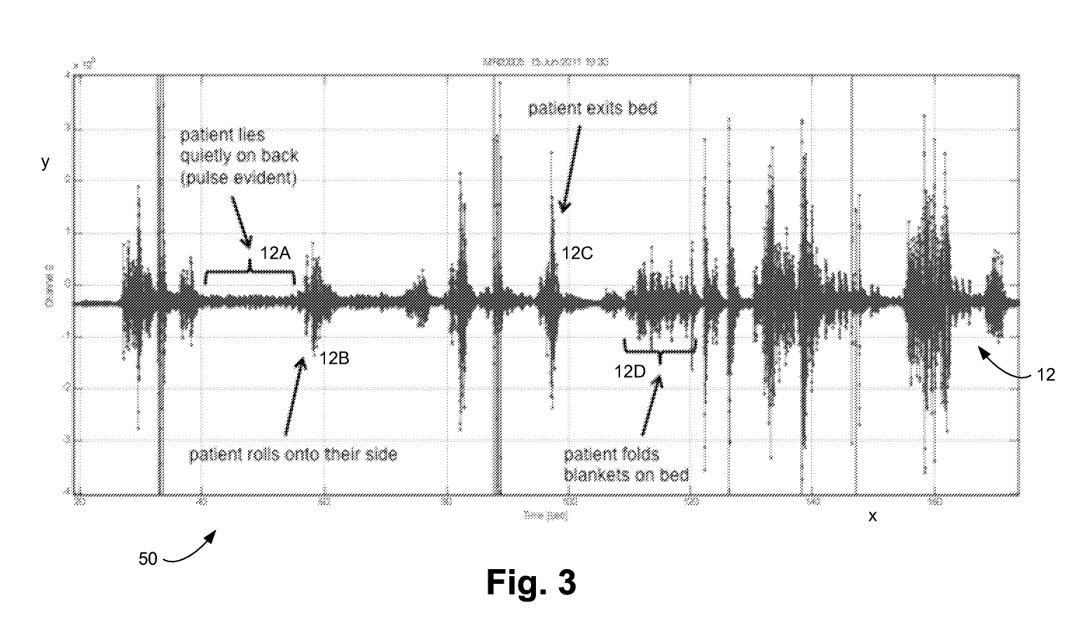

The functions of processing system 6 are described in additional detail with reference to FIG. 4, which is a flow chart illustrating one embodiment of a method for assessing physical stability of patient 2 using physiological data 12 captured by accelerometer 4.

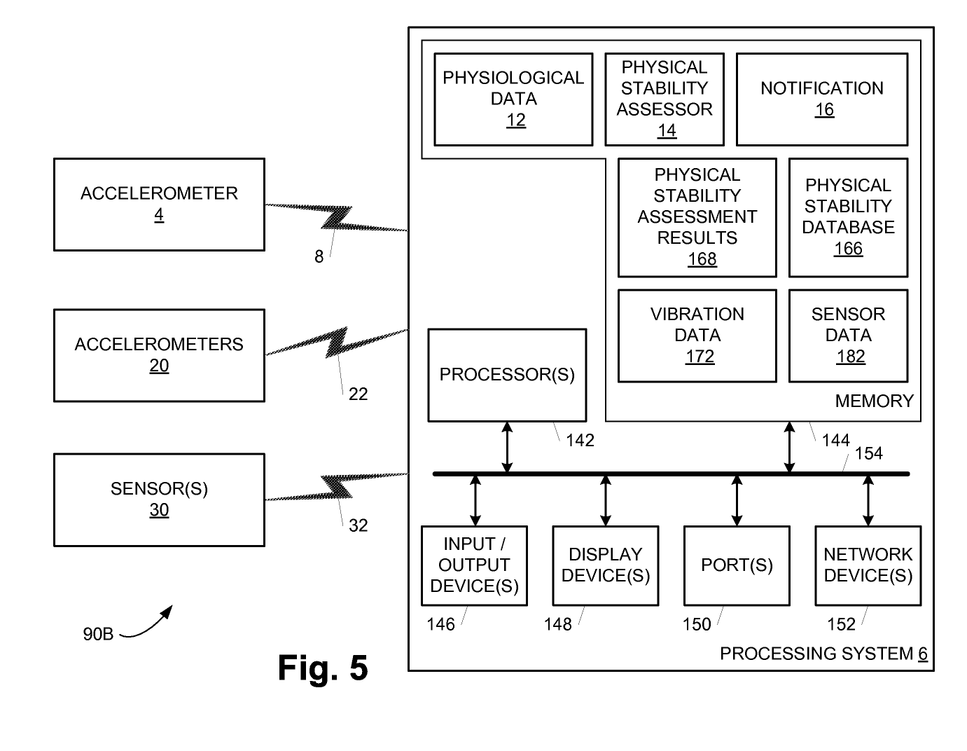

In FIG. 4, processing system 6 receives physiological data 12 from accelerometer 4 as indicated in a block 62. Processing system 140 may receive physiological data 12 as a continuous or periodic stream from accelerometer 4 or may retrieve the data by accessing the data from a computer readable medium (not shown) of the accelerometer 4. Processing system 6 assesses the physical stability 9 of patient 2 using physiological data 12 as indicated in a block 64. Processing system 6 may use frequency domain or time domain analysis to identify irregular motions of patient 2 in physiological data 12. Processing system 140 may also identify irregular motions by comparing and correlating known patterns from a physical stability database (e.g., physical stability database 166 shown in FIG. 1B) with motions of patient in physiological data 12. The physical stability database may include expected behavior of patient 2 derived from training or calibration processes as described in additional detail below. Processing system 6 may also detect irregular motions using physiological data 12, vibration data from other accelerometers disposed in proximity to patient 2 (e.g., vibration data 172 from accelerometers 20(1)-20(N) as shown in FIG. 5), and sensor data from other sensors (e.g., sensor data 182 from sensors 30 as shown in FIG. 5) in direct or indirect contact with patient 2.

Processing system 6 provides a notification 16 corresponding to the physical stability of patient 2 as indicated in a block 66. Processing system 6 may provide notification 16 to patient 2 or any suitable person associated with patient 2 such as healthcare professionals or family, friends, or others having a relationship with patient 2 in response to the physical stability 9 deviating from expected behavior of patient 2. Processing system 6 may provide notifications 16 at any suitable time and in any suitable way. For example, processing system 6 may provide an immediate notification 16 to patient 2 using any suitable audio and/or video device (not shown) (e.g., a verbal warning played through a sound device integrated with accelerometer 4 (not shown)) or a notification 16 that physical instability has been detected to an interested person. Processing system 6 may also store a log or other report of identified irregular motions and notifications 16 (e.g., physical stability assessment results 168 shown in FIG. 1B and 5) for later retrieval by an interested person. The log or other report may include comparisons with other patients with similar demographics whose physiological data has also been captured using an accelerometer 4 and stored in physical stability database 166. The notifications of processing system 6 may include conclusions reached based specific irregular motions detected by processing system 6. For example, a notification could note that a patient fell or is at a higher or changed risk of falling.

Processing system 6 may be configured according to a set of reporting policies that determine how notifications 16 are disseminated. For example, notifications 16 may be generated only when the detected irregular motions meet some statistically determined threshold with respect to physiological data 12 of patient 2 or the physiological data of other patients in physical stability database 166 captured using another accelerometer 4. The threshold may be set differently for different interested persons (e.g., a family member may receive a notification 16 at a lower threshold than a doctor).

FIG. 1B is a block diagram illustrating one embodiment of a processing environment 90A. Processing environment 90A includes accelerometer 4 in communication with processing system 6 across connection 8.

In the embodiment of FIG. 1B, accelerometer 4 includes three layers, or “wafers.” In particular, each accelerometer 4 includes a stator wafer 103, a rotor wafer 106, and a cap wafer 109. Stator wafer 103 includes electronics 113 that may be electrically coupled to various electrical components in rotor wafer 106 and cap wafer 109. Also, electronics 113 may provide output ports for coupling to electronic components external to accelerometer 4.

Rotor wafer 106 includes support 116 that is mechanically coupled to a proof mass 119. Although the cross-sectional view of accelerometer 4 is shown, according to one embodiment, support 116 as a portion of rotor wafer 106 surrounds proof mass 119. Consequently, in one embodiment, stator wafer 103, support 116, and cap wafer 109 form a pocket within which proof mass 119 is suspended.

Together, stator wafer 103, support 116, and cap wafer 109 provide a support structure to which proof mass 119 is attached via a compliant coupling. The compliant coupling may, in one embodiment, comprise high aspect ratio flexural suspension elements 123 described in U.S. Pat. No. 6,882,019.

Accelerometer 4 further includes a first electrode array 126 that is disposed on proof mass 119. In one embodiment, first electrode array 126 is located on a surface of proof mass 119 that is opposite the upper surface of stator wafer 103. The surface of the proof mass 119 upon which the first electrode array 126 is disposed is a substantially flat surface.

A second electrode array 129 is disposed on a surface of stator wafer 103 facing opposite first electrode array 126 disposed on proof mass 119. Because proof mass 126 is suspended over stator wafer 103, a substantially uniform gap 133 (denoted by d) is formed between first electrode array 126 and second electrode array 129. The distance d may comprise, for example, anywhere from 1 to 3 micrometers, or it may be another suitable distance.

Proof mass 119 is suspended above stator wafer 103 so that first electrode array 126 and second electrode array 129 substantially fall into planes that are parallel to each other and gap 133 is substantially uniform throughout the overlap between first and second electrode arrays 126 and 129. In other embodiments, electrode arrays 126 and 129 may be placed on other surfaces or structures of stator wafer 103 or proof mass 119.

High aspect ratio flexural suspension elements 123 offer a degree of compliance that allows proof mass 119 to move relative to the support structure (not shown) of accelerometer 4. Due to the design of flexural suspension elements 123, the displacement of proof mass 119 from a rest position is substantially restricted to a direction that is substantially parallel to second electrode array 129, which is disposed on the upper surface of stator wafer 103. Flexural suspension elements 123 are configured to allow for a predefined amount of movement of proof mass 119 in a direction parallel to second electrode array 129 such that gap 133 remains substantially uniform throughout the entire motion to the extent possible. The design of flexural suspension elements 123 provides for a minimum amount of motion of proof mass 119 in a direction orthogonal to second electrode array 129 while allowing a desired amount of motion in the direction parallel to second electrode array 129.

As proof mass 119 moves, capacitances between first and second electrode arrays 126 and 129 vary with the shifting of the arrays with respect to each other. Electronics 113 and/or external electronics are employed to detect or sense the degree of the change in the capacitances between electrode arrays 126 and 129. Based upon the change in the capacitances, such circuitry can generate appropriate signals that are proportional to the vibrations from patient 2 experienced by accelerometer 4.

The operation of accelerometer 4 is enhanced by the use of three-phase sensing and actuation as described by U.S. Pat. Nos. 6,882,019 and 7,484,411. Three-phase sensing uses an arrangement of sensing electrodes 126 and 129 and sensing electronics 113 to enhance the output signal of accelerometer 4 and allow for the sensitivity to be maximized in a desired range. It also allows the output of accelerometer 4 to be “reset” to zero electronically when the sensor is in any arbitrary orientation.

Processing system 6 represents any suitable processing device, or portion of a processing device, configured to implement the functions described herein. A processing device may be a laptop computer, a tablet computer, a desktop computer, a server, or another suitable type of computer system. A processing device may also be a mobile telephone with processing capabilities (i.e., a smart phone) or another suitable type of electronic device with processing capabilities. Processing capabilities refer to the ability of a device to execute instructions stored in a memory 144 with at least one processor 142. Processing system 6 represents one of a plurality of processing systems in a cloud computing environment in one embodiment.

Processing system 6 includes at least one processor 142 configured to execute machine readable instructions stored in a memory system 144. Processing system 6 may execute a basic input output system (BIOS), firmware, an operating system, a runtime execution environment, and/or other services and/or applications stored in memory 144 (not shown) that includes machine readable instructions that are executable by processors 142 to manage the components of processing system 6 and provide a set of functions that allow other programs to access and use the components. Processing system 6 stores physiological data 12 received from accelerometer 4 in memory system 144 along with physical stability assessor 14 that performs the method of FIG. 4 described above. Processing system 6 further stores physical stability database 166 and physical stability assessment results 168 in some embodiments.

Processing system 6 may also include any suitable number of input/output devices 146, display devices 148, ports 150, and/or network devices 152. Processors 142, memory system 144, input/output devices 146, display devices 148, ports 150, and network devices 152 communicate using a set of interconnections 154 that includes any suitable type, number, and/or configuration of controllers, buses, interfaces, and/or other wired or wireless connections. Components of processing system 6 (for example, processors 142, memory system 144, input/output devices 146, display devices 148, ports 150, network devices 152, and interconnections 154) may be contained in a common housing with accelerometer 4 (not shown) or in any suitable number of separate housings separate from accelerometer 4 (not shown).

Each processor 142 is configured to access and execute instructions stored in memory system 144 including physical stability assessor 14. Each processor 142 may execute the instructions in conjunction with or in response to information received from input/output devices 146, display devices 148, ports 150, and/or network devices 152. Each processor 142 is also configured to access and store data, including physiological data 12, physical stability database 166, and physical stability assessment results 168, in memory system 144.

Memory system 144 includes any suitable type, number, and configuration of volatile or non-volatile storage devices configured to store instructions and data. The storage devices of memory system 144 represent computer readable storage media that store computer-readable and computer-executable instructions including physical stability assessor 14. Memory system 144 stores instructions and data received from processors 142, input/output devices 146, display devices 148, ports 150, and network devices 152. Memory system 144 provides stored instructions and data to processors 142, input/output devices 146, display devices 148, ports 150, and network devices 152. Examples of storage devices in memory system 144 include hard disk drives, random access memory (RAM), read only memory (ROM), flash memory drives and cards, and other suitable types of magnetic and/or optical disks.

Input/output devices 146 include any suitable type, number, and configuration of input/output devices configured to input instructions and/or data from a user to processing system 6 and output instructions and/or data from processing system 6 to the user. Examples of input/output devices 146 include a touchscreen, buttons, dials, knobs, switches, a keyboard, a mouse, and a touchpad.

Display devices 148 include any suitable type, number, and configuration of display devices configured to output image, textual, and/or graphical information to a user of processing system 6. Examples of display devices 148 include a display screen, a monitor, and a projector.

Ports 150 include suitable type, number, and configuration of ports configured to input instructions and/or data from another device (not shown) to processing system 6 and output instructions and/or data from processing system 6 to another device.

Network devices 152 include any suitable type, number, and/or configuration of network devices configured to allow processing system 6 to communicate across one or more wired or wireless networks (not shown). Network devices 152 may operate according to any suitable networking protocol and/or configuration to allow information to be transmitted by processing system 6 to a network or received by processing system 152 from a network.

Connection 8 includes any suitable type and combination of wired and/or wireless connections that allow accelerometer 4 to provide physiological data 12 to processing system 6. Connection 22 may connect to one or more ports 150 and/or one or more network devices 152 of processing system 6. For example, connection 8 may comprise a wireless network connection that includes a wireless network device (not shown) that transmits physiological data 12 from accelerometer 4 to processing system 6. As another example, connection 8 may comprise a cable connected from accelerometer 4 to a port 150 to transmit physiological data 12 from accelerometer 4 to processing system 6.

Referring back to FIGS. 1A-1B, an optimal placement for patient 2 to wear accelerometer 4 may be determined using various methods to enhance the detectability of physiological data 12 by accelerometer 4. In at least some of the methods, accelerometer 4 is initially affixed at one location on patient 2 and then signals are observed at accelerometer 4. Accelerometer 4 is then affixed at one or more different locations on patient 2 and the signals observed again at accelerometer 4. An adjustable clamp (not shown) may be used to temporarily affix accelerometer 4 to patient.

Accelerometer 4 and processing system 6 may also be used to capture of additional information for calibrating or training activity detection unit 164 according to various techniques. Patients may be asked to perform a series of activities that include activities of interest such as walking, standing up from a chair, or getting out of bed. Accelerometer 4 captures physiological data 12 of these activities to allow processing system 6 to correlate physiological data 12 with the activities in activity database 166.

FIG. 5 is a block diagram illustrating one embodiment of a processing environment 90B for assessing physical stability of patient 2 using multiple accelerometers 4 and 20 and/or other sensors 30. As with processing environment 90A in FIG. 1B, processing environment 90B includes accelerometer 4 worn by patient 2 in communication with processing system 6 across connection 8. Processing environment 90B also includes one or more additional accelerometers 20 in communication with processing system 6 across one or more connections 22 and/or one or more sensors 30 in communication with processing system 6 across one or more connections 32.

Accelerometers 20 capture vibration data 172 in an area of patient 2 and transmit vibration data 172 to processing system 6 using a connection 22. The area of patient 2 includes a patient environment, which, as used herein, refers to a bed, chair, wheelchair, an examination table, or other suitable apparatus with one or more support surfaces configured for a patient to assume a relatively stationary position (e.g., lying and/or sitting). The area of patient 2 also includes other apparatus and structures in proximity to patient 2 such as the wall, floor, or other furniture or items.

Accelerometers 20 may be mounted or otherwise disposed on a patient environment to monitor and detect various aspects of the health and condition of patient 2 in addition to the physical stability of patient 2. These accelerometers 20 detect vibration data 172 of patient 2 that transfers from patient 2 to the accelerometers 20 through the patient environment (e.g., through the bed, chair, or wheelchair) rather than through a direct connection from patient 2 to accelerometers 20. Processing system 6 may detect and/or infer specific features of the physiological condition of patient 2 from vibration data 172.

Accelerometers 20 may also be disposed on multiple apparatus and/or structures in multiple locations in the area of a patient. These accelerometers 20 each provide vibration data 172 to processing system 6 which extracts information for detecting activities of patient 2 or others in the area that may not otherwise be detectable using an individual accelerometer 4 or 20. The term activities refers to the actions of one or more patients 2 or others in or near an area that generate vibrations that transmit through materials in the area to one or more accelerometers 20. The materials include structural elements that form the area such as a floor, walls, a ceiling, windows, and doors, materials that comprise patient environments, and other objects (e.g., medical equipment or home furnishings) present in the area. These accelerometers 20 form a data network that enables processing system 6 to correlate and analyze vibration data 172 from accelerometers 20 simultaneously. The activities detected from vibration data 172 by processing system 6 supports the care of patients 2 where the system is employed.

Sensors 30, if present, capture sensor data 182 from patient 2 and/or the area of patient 2 and transmit sensor data 182 to processing system 6 using a connection 32. Sensors 30 may be placed in direct or indirect contact with patient 2 to generate sensor data 182. Processing system 6 may use the sensor data in conjunction with physiological data 12 and/or vibration data 172 to assess the physical stability of patient 2.

Processing system 6 may process vibration data 172 and/or sensor data 182 along with physiological data 12 as described above to further assess a physical stability 9 of patient 2.

The above embodiments may advantageously enable non-intrusive, continuous, long-term, inexpensive assessments of the physical stability of patients which may otherwise be difficult or time consuming for health care professionals to make. The embodiments may also provide the ability to communicate results to interested persons based on configurable policies. This may allow the above embodiments to participate in health care processes for patients in an active and timely manner. As a result, patients may remain in their own homes longer, thereby increasing their quality of life, and incurring less institutional care.

What is claimed is:

1. A system comprising:
an accelerometer worn by a patient to capture physiological data of the patient and provide the physiological data; and
a processing system to receive the physiological data and assess a physical stability of the patient using the physiological data.

2. The system of claim 1 wherein the processing system is to generate a notification corresponding to the physical stability of the patient.

3. The system of claim 2 wherein the processing system is to generate the notification in response to determining that the physical stability indicates a potential for the patient to fall.

4. The system of claim 2 wherein the processing system is to provide the notification to the patient.

5. The system of claim 2 wherein the processing system is to provide the notification to a person associated with the patient.

6. The system of claim 1 wherein the physiological data represents internal and external body movements of the patient.

7. The system of claim 1 wherein the accelerometer includes a proof mass with a first electrode array suspended above a second electrode array disposed on a wafer.

8. The system of claim 1 wherein the accelerometer includes three-phase sensing and actuation.

9. The system of claim 1 wherein the accelerometer detects changes in capacitances between a first electrode arrays disposed on a proof mass and a second electrode array disposed on a wafer.

10. A method performed by a processing system, the method comprising:
receiving physiological data from an accelerometer worn by a patient using the processing system;
processing the physiological data to assess a physical stability of the patient based on the physiological data using the processing system; and
providing a notification corresponding to the physical stability to the patient using the processing system.

11. The method of claim 10 further comprising:
providing the notification to a person associated with the patient.

12. The method of claim 10 further comprising:
comparing the physical stability to an expected behavior of a patient; and
providing the notification in response to the physical stability deviating from the expected behavior.

13. A computer-readable storage medium storing instructions that, when executed by a processing system, perform a method comprising:
receiving physiological data from a first accelerometer worn by a patient;
receiving vibration data from a second accelerometer in an area of the patient;
processing the physiological data and the vibration data to assess a physical stability of the patient based on the physiological data; and
providing a notification corresponding to the physical stability to the patient.

14. The computer-readable storage medium of claim 13, wherein the second accelerometer is mounted to a patient environment.

15. The computer-readable storage medium of claim 13, wherein the physiological data represents internal and external body movements of the patient.
